(19) United States
(12) Patent Application Publication
(10) Pub. No.: US 2024/0239930 A1
(43) Pub. Date: Jul. 18, 2024

(54) FUNCTIONALIZED MALEIC ACID COPOLYMERS FOR ENHANCED BIOACTIVITY

(71) Applicant: UNIVERSITY OF TENNESSEE RESEARCH FOUNDATION, Knoxville, TN (US)

(72) Inventors: Nathan G. BRADY, Knoxville, TN (US); Cameron E. WORKMAN, Knoxville, TN (US); Barry D. BRUCE, Knoxville, TN (US); Brain K. LONG, Knoxville, TN (US)

(73) Assignee: UNIVERSITY OF TENNESSEE RESEARCH FOUNDATION, Knoxville, TN (US)

(21) Appl. No.: 18/558,846

(22) PCT Filed: May 6, 2022
(86) PCT No.: PCT/US22/28113

§ 371 Date: Nov. 3, 2023

Related U.S. Application Data
(60) Provisional application No. 63/185,252, filed on May 6, 2021.

(51) Int. Cl. C08F 212/08; A61K 9/127

(52) U.S. Cl. CPC C08F 212/08; A61K 9/1273

(57) ABSTRACT

Modified maleic acid copolymers have an esterified styrene maleic acid or diisobutylene maleic acid. The copolymers have 1% to 90% total esterification of the maleic acid (MA), thereby having an ester of Y¹ and/or Y² of the general formula —OR¹. R¹ is a moiety present in enough units of the copolymer in one or both of Y¹ and Y² to provide a preselected percent of total esterification, and if either or both of Y¹ and Y² are not esterified, it is a hydrogen or a carboxylate unit with a counterion X⁺. R¹ comprises (i) a linear alkane chain, (ii) a linear chain alkoxy alkane of the formula —(CH₂)_{q}O(CH₂)ᵣCH₃, (iii) an alkane containing or terminating with a cyclic carbon chain, (iv) an alkoxy alkane containing or terminating with a cyclic carbon chain, (v) a chain containing a repeating sequence of (CH₂CH₂O)ₜ terminating with —OR², or a mixture of (i) to (v).

### RELATED APPLICATIONS

This application claims the benefit of U.S. Provisional Application No. 63/185,252, filed May 6, 2021, the entirety of which is incorporated herein by reference.

### TECHNICAL FIELD

This application relates to modified maleic acid copolymers, more particular, to modified styrene maleic acid copolymers and diisobutylene maleic acid copolymers having the maleic acid hydrolyzed and esterified with 1% to 90% total esterification with (i) a linear alkane chain having 5 or more carbons, (ii) a linear chain alkoxy alkane of the formula —(CH₂)_{q}O(CH₂)ᵣCH₃ where q is 1 to 5 and r is 1 to 15, but styrene maleic acid copolymers have the proviso that when q=2, r is not 3, or (iii) a chain containing a repeating sequence of (CH₂CH₂O)ₜ terminating with —OR² wherein t equals a value of 1 to 50 and R² is hydrogen, any linear or cyclic alkane, or any mixture of (i) to (iii).

### BACKGROUND

Over the past decade, styrene-maleic acid copolymers (SMAs) have become widely used for the solubilization of membrane proteins. Diisobutylene-maleic acid copolymers (DIBMAs) likewise have been studied for their ability to solubilize membrane proteins. Their unique ability to form styrene-maleic acid lipid particles (SMALPs) and Diisobutylene maleic acid lipid particles (DIBMALPs), which are polymer bound nanodiscs comprised of protein within an annulus of retained native lipids, has made these amphiphilic copolymers the subject of numerous investigations. Salient reports include studies designed to understand how various SMA molecular characteristics and DIBMA molecular characteristics play into SMALP formation and efficacy of these polymers in protein extraction. For example, it has been shown in literature that both the molecular weight of the polymer and the incorporation ratio of the monomeric units are both crucial parameters in this process.

Despite these advances, little is known about the mechanism of SMA-facilitated protein extraction and solubilization. Many studies designed to probe this mechanism have shown that altering the chemical composition of SMA can result in characteristic changes in nanodisc formation. Examples of such investigations include studies by Ramamoorthy and coworkers in which the maleic anhydride repeating units of SMA copolymers were converted into maleimides bearing quaternary ammonium pendant groups, increasing the polymer's tolerance to divalent cations and low pH. Ravula et al., pH Tunable and Divalent Metal Ion Tolerant Polymer Lipid Nanodiscs. Langmuir 2017, 33 (40), 10655-10662. In another study, Konkolewicz and Lorigan achieved a similar result by performing esterification and amidation reactions on SMAs using a variety of moieties, ranging from glucose to 2-aminothanol. Burridge et al., Simple Derivatization of RAFT-Synthesized Styrene-Maleic Anhydride Copolymers for Lipid Disk Formulations. Biomacromolecules 2020, 21 (3), 1274-1284. Overduin and coworkers showed that SMA functionalization can be used to manipulate the size of resulting SMALPs, further highlighting how functionalized SMA samples can be utilized to alter various aspects of the protein extraction process. Esmaili et al., The effect of hydrophobic alkyl sidechains on size and solution behaviors of nanodiscs formed by alternating styrene maleamic copolymer, Biochimica et Biophysica Acta (BBA)—Biomembranes 2020, 1862 (10), 183360. Though all of these studies demonstrate the effects of SMA functionalization, they do not investigate how SMA functionalization alters extraction efficiency or selectivity in specific protein solubilization trials. An understanding of the fundamental relationships between specific SMA derivatization and the resultant extraction selectivity and/or efficiency could facilitate the selection and design of SMAs with enhanced extraction yields, as well as the potential to target specific proteins.

Several of the inventors hereof have recently shown that certain commercially available SMA-based copolymers exhibit the ability to form SMALPs from chloroplast thylakoid membranes, which is of note due to a lack of studies investigating galactolipid rich membranes of cyanobacterial thylakoid membranes. Korotych et al., Poly(styrene-co-maleic acid)-mediated isolation of supramolecular membrane protein complexes from plant thylakoids, Biochimica et Biophysica Acta (BBA)—Bioenergetics 2021, 1862 (3), 148347. The commercially available SMA studies was SMA 1440 (Cray Valley, now available from Polyscope), a 1.5:1 styrene:maleic acid copolymer that is monoesterified with butoxyethanol allegedly to 72% monoesterification (36% total esterification), appears to selectively extract the Photosystem I (PSI) trimer from the thylakoid membranes of Thermosynechococcus elongatus (Te), whereas the most widely used SMA copolymers, those containing a 2:1 ratio of styrene:maleic acid, are ineffective at solubilizing thylakoid membranes from Te. Brady et al., Non-detergent isolation of a cyanobacterial photosystem I using styrene maleic acid alternating copolymers. RSC Advances 2019, 9 (54), 31781-31796. This is of interest because the non-esterified version of the same SMA, PRO 10235, shows very little activity in the extraction of PSI from thylakoid membranes in Te. In addition, PRO 10235 demonstrated the lowest extraction activity of the five tested SMAs using thylakoids from pea and spinach chloroplasts, suggesting a generally lower activity with galactolipid-rich membranes. We hypothesized that the butoxyethanol esterification of SMA 1440 must be critical for the insertion into the galactolipid-rich thylakoid membranes of cyanobacteria.

### SUMMARY

In one aspect, modified maleic acid copolymers herein are of a general formula II and having 1 to 90% total esterification of the maleic acid (MA).

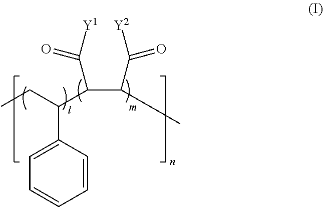

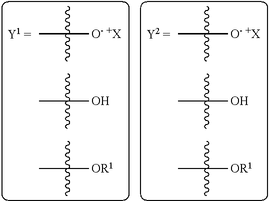

R¹ is a moiety present in enough m units in one or both of Y¹ and Y² to provide a preselected percent of total esterification, and if not esterified are a hydrogen or a carboxylate unit with a counterion X⁺. R¹ is selected from the group consisting of (i) a linear alkane chain, (ii) a linear chain alkoxy alkane of the formula —(CH₂)_{q}O(CH₂)ᵣCH₃ where q is 1 to 5 and r is 1 to 15, (iii) an alkane containing or terminating with a cyclic carbon chain, (iv) an alkoxy alkane containing or terminating with a cyclic carbon chain, (v) a chain containing a repeating sequence of (CH₂CH₂O)ₜ terminating with —OR² wherein t equals a value of 1 to 50 and R² is hydrogen, a linear alkane, or a cyclic alkane, and mixtures thereof. 1 and m have a ratio in a range of 0.5:1 to 8:1, and n yields a copolymer having an average molecular weight of less than 500,000 daltons.

In some embodiments, R¹ comprises the linear alkane chain and/or the linear chain alkoxy alkane, which can be partially or fully halogenated or partially or fully deuterated. In any embodiment having a cyclic carbon, the cyclic carbon chain can be partially or fully halogenated or partially or fully deuterated.

In all embodiments, R¹ can be the same in all esterified n units or can be different in a plurality of the esterified n units. In one embodiment, R¹ comprises the linear chain alkoxy alkane of the formula —(CH₂)_{q}O(CH₂)ᵣCH₃, q=1 to 5, and r=1 to 10.

In one embodiment, R¹ comprises the chain containing a repeating sequence of (CH₂CH₂O)ₜ terminating with —OR². In some embodiments, t=1 to 10 and the copolymer is monoesterified with greater than 20% total esterification. In one embodiment, t=2 and the copolymer is monoesterified with about 10 to 15% total esterification and t=2.

In one embodiment, the copolymer is monoesterified with greater than 10% total esterification, and R¹ comprises (ii) and r is 5 to 15. In another embodiment, the copolymer is monoesterified with greater than 20% total esterification, and R¹ comprises (ii) and r is 9 to 15.

In another aspect, modified maleic acid copolymer herein are of a general formula II and having 1 to 90% total esterification of the maleic acid (MA).

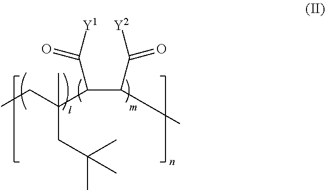

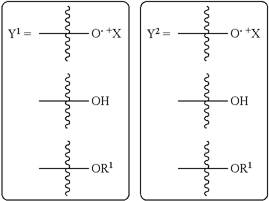

R¹ is a moiety present in enough m units in one or both of Y¹ and Y² to provide a preselected percent of total esterification, and if not esterified are a hydrogen or a carboxylate unit with a counterion X⁺. R¹ is selected from the group consisting of (i) a linear alkane chain, (ii) a linear chain alkoxy alkane of the formula —(CH₂)_{q}O(CH₂)ᵣCH₃ where q is 1 to 5 and r is 1 to 15, (iii) an alkane containing or terminating with a cyclic carbon chain, (iv) an alkoxy alkane containing or terminating with a cyclic carbon chain, (v) a chain containing a repeating sequence of (CH₂CH₂O)ₜ terminating with —OR² wherein t equals a value of 1 to 50 and R² is hydrogen, a linear alkane, or a cyclic alkane, and mixtures thereof. 1 and m have a ratio in a range of 0.5:1 to 8:1, and n yields a copolymer having an average molecular weight of less than 500,000 daltons.

In some embodiments, R¹ comprises the linear alkane chain and/or the linear chain alkoxy alkane, which can be partially or fully halogenated or partially or fully deuterated. In any embodiment having a cyclic carbon, the cyclic carbon chain can be partially or fully halogenated or partially or fully deuterated.

In all embodiments, R¹ can be the same in all esterified n units or can be different in a plurality of the esterified n units. In one embodiment, R¹ comprises the linear chain alkoxy alkane of the formula —(CH₂)_{q}O(CH₂)ᵣCH₃, q=1 to 5, and r=1 to 10.

In one embodiment, R¹ comprises the chain containing a repeating sequence of (CH₂CH₂O)ₜ terminating with —OR². In some embodiments, t=1 to 10 and the copolymer is monoesterified with greater than 20% total esterification. In one embodiment, t=2 and the copolymer is monoesterified with about 10 to 15% total esterification and t=2.

In one embodiment, the copolymer is monoesterified with greater than 10% total esterification, and R¹ comprises (ii) and r is 5 to 15. In another embodiment, the copolymer is monoesterified with greater than 20% total esterification, and R¹ comprises (ii) and r is 9 to 15.

In yet another aspect, modified maleic acid copolymer lipid particles herein have a lipid from a phospholipid rich membrane or a galactolipid rich membrane and a modified maleic acid copolymer of any of the chemical structures disclosed herein. In one embodiment, the lipid is from a galactolipid rich membrane of a cyanobacterium, which can be Thermosynechococcus elongatus.

### BRIEF DESCRIPTION OF THE DRAWINGS

FIG. 1A is an illustrative representation of SMA extraction of proteins from a PSI lipid membrane.

FIG. 1B is an illustrative representation of SMA-functionalized with butoxyethanol for the extraction of a protein from a PSI lipid membrane.

FIG. 1C is an illustrative representation of SMA-functionalized with dodecoxyethanol for the extraction of a protein from a PSI lipid membrane.

FIG. 2 is a bar graph of the pH below which various SMA-functionalized polymers of varying monoesterification aggregate and precipitate from solution.

FIG. 3 is a bar graph of the concentration of magnesium ions above which various SMA-functionalized polymers of varying monoesterification aggregate and precipitate from solution.

FIG. 5 is a graph showing a comparison of percent solubilization efficiency (SE) of chlorophyll and chlorophyll-containing complexes from a thylakoid membrane as a function of percent monoesterification for each SMA-functionalized polymer tested.

FIG. 6 is a graph showing a comparison of percent solubilization efficiency (SE) of chlorophyll and chlorophyll-containing complexes from a thylakoid membrane as a function of percent monoesterification for butoxyethanol-functionalized SMA and a DEG-functionalized SMA each comprising a total of seven carbon or carbon and oxygen atoms in their sidechain.

FIG. 7 is a graph showing a comparison of percent solubilization efficiency (SE) of chlorophyll and chlorophyll-containing complexes of a PSI membrane as a function of percent esterification for decoxyethanol-functionalized SMA and a TEG-functionalized SMA each comprising a total of thirteen carbon or carbon and oxygen atoms in their sidechain.

FIG. 8 is the plot of esterified SMA solubilization efficiency from thylakoid membranes as a function of the average number of carboxylates per unfunctionalized carboxylate moiety.

FIG. 9 is a bar graph of solubilization efficiency from thylakoid membranes of various modified SMA copolymers having about 30% monoesterification.

FIG. 10 is a bar graph of solubilization efficiency from thylakoid membranes of various modified SMA copolymers having greater than 45% monoesterification.

FIG. 11 is a photograph of results for sucrose density gradients of PSI-SMALPs.

FIG. 12 is a TEM image PSI-SMALPs from Te using SMA-Dodec-(52).

FIG. 13 is a TEM image PSI-SMALPs from Te using SMA-Oct-(59).

FIG. 14 is a TEM image PSI-SMALPs from Te using SMA-Hex-(53).

FIG. 15 is a TEM image PSI-SMALPs from Te using SMA-Dec-(60).

FIG. 16 is a TEM image PSI-SMALPs from Te using SMA-1440.

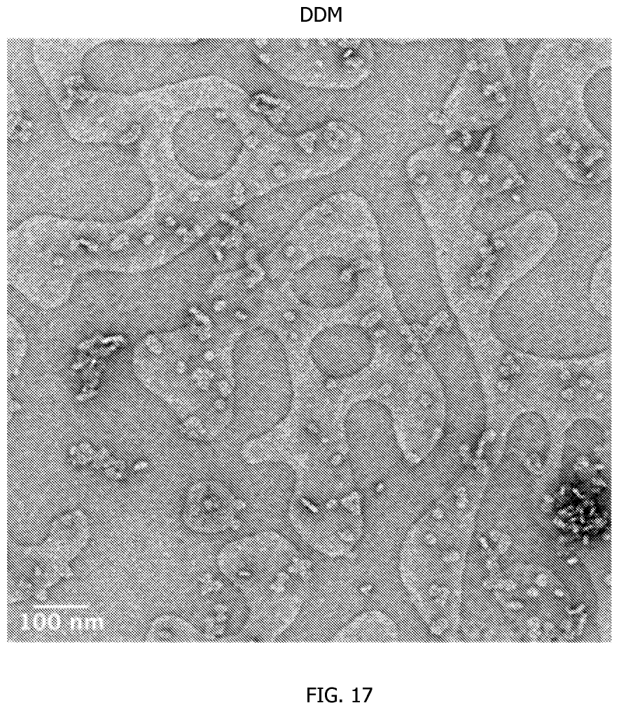

FIG. 17 is a TEM image PSI-SMALPs from Te using SMA-DDM.

### DETAILED DESCRIPTION

The following description and drawings are illustrative and are not to be construed as limiting. Numerous specific details are described to provide a thorough understanding of the disclosure. In certain instances, however, well-known or conventional details are not described to avoid obscuring the description. References to one or an embodiment in the present disclosure can be, but not necessarily, are references to the same embodiment; and, such references mean at least one of the embodiments.

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710) and other similar references. As used herein, the singular forms “a,” “an,” and “the,” refer to both the singular as well as plural, unless the context clearly indicates otherwise. The abbreviation, “e.g.” is derived from the Latin exempli gratia and is used herein to indicate a non-limiting example. Thus, the abbreviation “e.g.” is synonymous with the term “for example.” As used herein, the term “comprises” means “includes.” All publications, patent applications, patents, and other references mentioned herein are expressly incorporated herein by reference in their entirety.

“Isolated” as used herein refers to biological proteins that are removed from their natural environment and are isolated or separated and are free from other components with which they are naturally associated. The term “purified” does not require absolute purity; rather, it is intended as a relative term. Thus, for example, a purified or “substantially pure” protein preparation is one in which the protein referred to is more pure than the protein in its natural environment within a cell or within a production reaction chamber (as appropriate).

As used herein, relative terms, such as “substantially,” “generally,” “approximately,” “about,” and the like are used herein to represent an inherent degree of uncertainty that can be attributed to any quantitative comparison, value, measurement, or other representation. These terms are also utilized herein to represent the degree by which a quantitative representation can vary from a stated reference without resulting in a change in the basic function of the subject matter at issue. In certain example embodiments, the term “about” is understood as within a range of normal tolerance in the art for a given measurement, for example, such as within 2 standard deviations of the mean. In certain example embodiments, depending on the measurement “about” as used herein means said value±3 when the value is expressed as a percentage and +5% of a value when expressed as or based on a measurement and it is not a percentage. Unless otherwise clear from context, all numerical values provided herein can be modified by the term about. “Substantially free” or “free” besides the values just stated, can be almost zero or zero, respectively.

In one aspect, modified maleic acid copolymers of general formula I, i.e., styrene maleic acid copolymers, that have a total esterification of 1% to 90% of the maleic acid (MA) and have R¹ as a moiety present in enough m units in one or both of Y¹ and Y² to provide a preselected percent of total esterification within this range are disclosed.

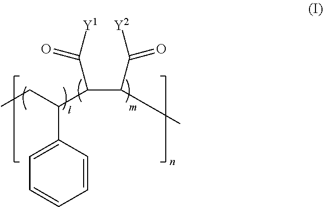

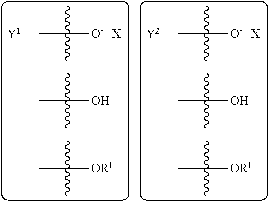

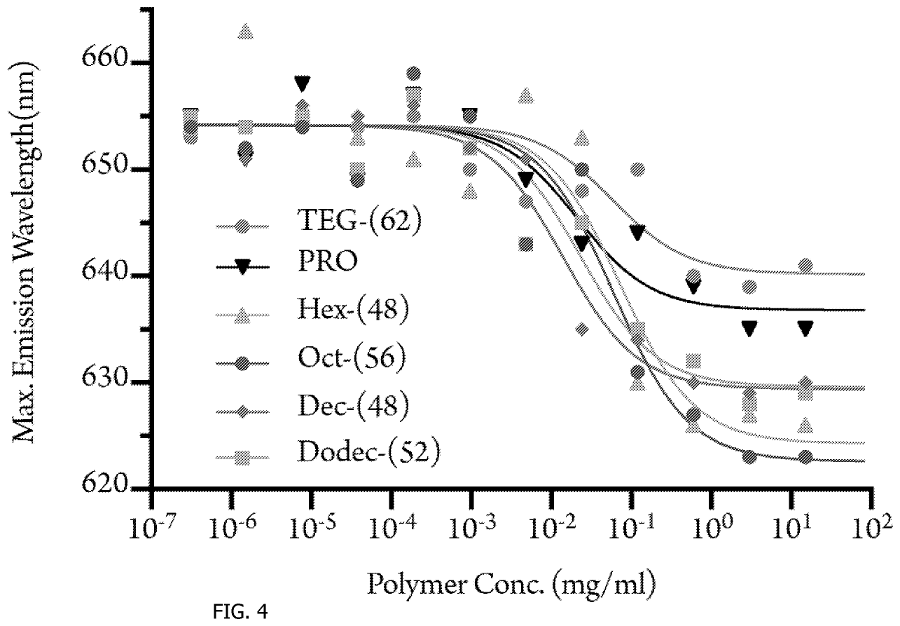

In one embodiment, the total esterification is in the range of 5% to 80% total esterification, more preferably in a range of 10% to 75%, and even more preferably in a range of 22% to 50%. In the examples, the preselected percent is reported as the percent of monoesterification. The total esterification is half of the value reported for the percent of monoesterification. For example, in FIG. 2 the greatest monoesterification is 56% when —OR¹ is octoxyl ethoxylate, which is a total esterification of 28%. Any Y¹ and Y² that are not esterified are a hydrogen or a carboxylate unit (—O—) with a general counterion X⁺. X⁺ is selected from the group consisting of ammonium, lithium, sodium, and potassium ions.

R¹ comprises (i) a linear alkane chain having 5, 7, 9, or 11 or more carbons, and optionally terminating with or containing a cycloalkane or cyclic ether, (ii) a linear chain alkoxy alkane of the formula —(CH₂)_{q}O(CH₂)ᵣCH₃ where q is 1 to 5 and r is 1 to 15, with the proviso that when r=2, q is not 3, i.e., —OR¹ is not butyloxy ethoxylate, and optionally terminating with or containing a cycloalkane in the r segment, or (iii) a chain containing a repeating sequence of (CH₂CH₂O)ₜ terminating with —OR² wherein t equals a value of 1 to 50 and R² is hydrogen or any linear alkane, or cyclic alkane, or any mixture thereof.

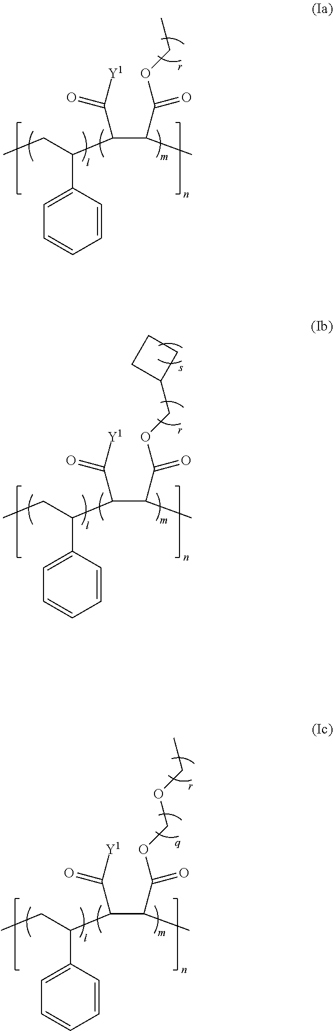

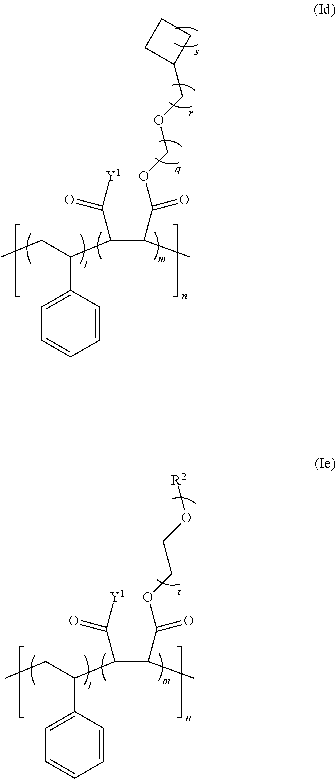

In some embodiments, R¹ is the same in all esterified m units. In other embodiments, R¹ is different in a plurality of the esterified m units. In most embodiments the copolymer is monoesterified and has greater than 10% or greater than 15%, or even greater than 25% total esterification.

In formula (Ia), r can be 4, 6, 8, 10 or more carbons, but is typically less than or equal to 20. In some embodiments, r is 10 to 25, more preferably 10 to 20.

In formula (Ib), r can be any integer from 0 to 12. In some embodiments, r is 5 to 25, more preferably 8 to 20, and s is 0 to 9.

In formula (Ic), q is typically 1 to 5 and r is 1 to 15. In some embodiments, q is 2 to 5, but when q=2, r is not 3. In other embodiments, q is 2 to 5 and r is 5 to 15, or even 8 to 15. Several examples for —OR₁ are hexoxy ethoxylate, heptoxy ethoxylate, octoxy ethoxylate, decoxy ethoxylate, undecoxy ethoxylate, or dodecoxy ethoxylate. In an embodiment where —OR₁ is octoxy ethoxylate and the copolymer is about or greater than 50% monoesterified, the functionalized copolymer is stable in aqueous solution at a pH greater than 7.0 and at a magnesium ion concentration less than 10 mM. In an embodiment where R¹ is decoxy ethoxylate and the copolymer is greater than 30% monoesterified, the functionalized copolymer is stable in aqueous solution at a pH greater than 6.5 and at a magnesium ion concentration less than 10 mM.

In formula (Id), q is typically 1 to 5 and r is 1 to 15 and s is typically 0 to 9. In some embodiments, q is 2 to 5 and r is 5 to 15, or even 8 to 15.

In one embodiment, R¹ comprises the linear alkane chain and/or the linear chain alkoxy alkane, which can be partially or fully halogenated or partially or fully deuterated. Likewise, if a cyclic carbon chain is present on either of the linear alkane chain and/or the linear chain alkoxy alkane, the carbons here can also be partially or fully halogenated or partially or fully deuterated.

Referring now to formula (Ie), R¹ comprises the chain containing a repeating sequence of (CH₂CH₂O)ₜ terminating with —OR², the copolymer is monoesterified with greater than 20% total esterification, and t=1 to 50. R² can be hydrogen, any linear alkane, or cyclic alkane. When R² is a linear alkane, the number of carbon atoms can be 1 to 16. When R² is a cyclic alkane, the number of carbon atoms in the ring is 3 to 12. Several examples are diethylene glycol, triethylene glycol, tetraethylene glycol, pentaethylene glycol, and hexaethylene glycol. In an embodiment where —OR¹ is diethylene glycol and the copolymer is greater than 50% monoesterified, the functionalized copolymer is stable in aqueous solution at a pH greater than 5.0 and at a magnesium ion concentration up to 50 mM. In an embodiment where R¹ is tetraethylene glycol and the copolymer is greater than 40% monoesterified, the functionalized copolymer is stable in aqueous solution at a pH greater than 5.0 and at a magnesium ion concentration up to 100 mM.

The modified maleic acid copolymers are generally unlimited in the ratio of 1 to m and in the size of n, which determines the molecular weight thereof. The average molecular weight (M_{w}) will typically be less than 500,000 daltons (Da), more particularly less than 150,000 Da. In some embodiments, the average molecular weight is less than 20,000 Da but greater than 1,500 Da. M_{w}/Mₙ(Mₙ being the number average molecular weight) indicates the polydispersity, and will typically be less than 10, more particularly less than 4, or even less than 3. In some embodiments, the polydispersity will be less than 2 (for example less than 1.5).

Numerous styrene/maleic anhydride copolymers are commercially available from Sartomer Inc. and Cray Valley HSC (Polyscope), and are identified by the base resins SMA 1000, SMA 2000, SMA 3000 and SMA 4000, etc. In the case of SMA 1000, SMA 2000, SMA 3000 and SMA 4000 the ratio of styrene to maleic anhydride is to 1:1, 2:1, 3:1 and 4:1, respectively. In these instances, the styrene forms an increasing number of short blocks as the styrene content is increased. SMA 2000, SMA 3000 and SMA 4000 are available as powder, flake or ultrafine powder preparations. Typical molecular weights for SMA 2000 are M_{w} 7,500 (Mₙ 2,700); for SMA3 000 are M_{w} 9,500 (Mₙ 3,050) and for SMA 4000 are M_{w} 11,000 (Mₙ 3,600) as assessed by gel permeation chromatography (GPC). Additionally, the base resin is available as ester or imide derivatives thereof. Example ester derivatives include SMA 1440 (Mₙ 2900), SMA 17352 (Mₙ 2900), SMA 2625 (Mₙ 3100), SMA 3840 (Mₙ 4100). Example imide derivatives include SMA 10001 (Mₙ 2100), SMA 20001 (Mₙ 2700), SMA 30001 (Mₙ 3050), SMA 40001 (Mₙ 3600). These base resins can be esterified to form a water-soluble salt.

The 1 to m monomer ratio of styrene to maleic acid can be in a range of 1:1 to 8:1. Exemplary monomer ratios herein are typically greater than 1:1 and may include but are not limited to 1.2:1, 1.3:1, 1.5:1, 2:1, 2.5:1, 3:1, 3.5:1, 4:1, 4.5:1, 6:1. In one embodiment, the copolymer of styrene and maleic acid (or salt thereof) has an average molecular weight in the range 1,000 to 12,000 and a ratio of styrene to maleic acid of greater than 1:1.

In another aspect, modified maleic acid copolymers of general formula II, i.e., diisobutylene maleic acid (DIBMA) copolymers, that have a total esterification of 1% to 90% of the maleic acid (MA) and have R¹ as a moiety present in enough m units in one or both of Y¹ and Y² to provide a preselected percent of total esterification within this range are disclosed.

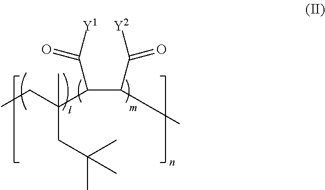

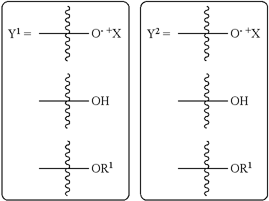

In one embodiment, the total esterification is in the range of 5% to 80% total esterification, more preferably in a range of 10% to 75%, and even more preferably in a range of 22% to 50%. Any Y¹ and Y² that are not esterified are a hydrogen or a carboxylate unit —O⁻ with a general counterion X⁺. X⁺ can be any of the ions noted above with respect to formula I.

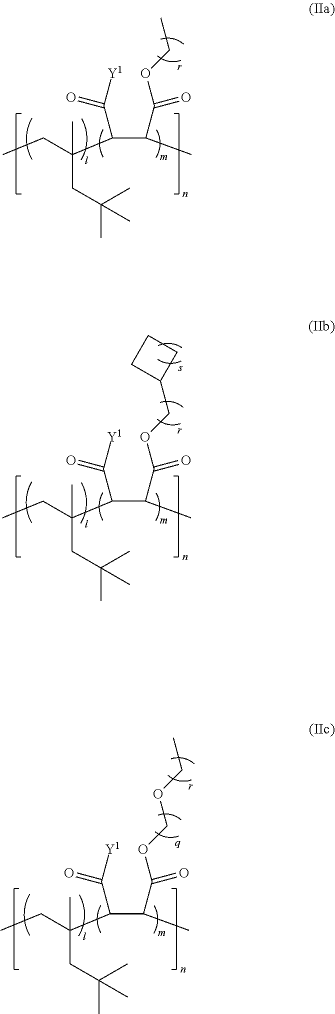

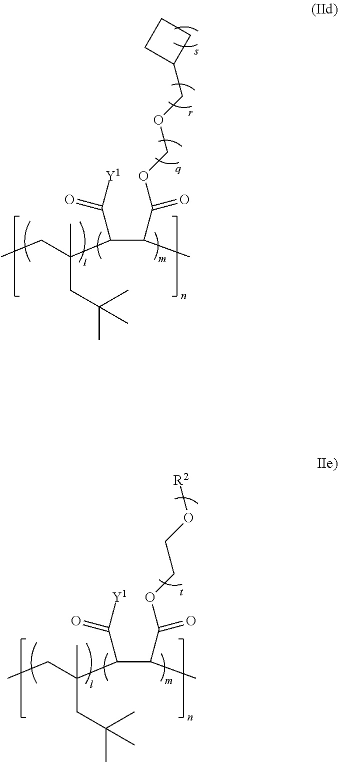

R¹ comprises (i) a linear alkane chain having 1 or more carbons, more preferably 4 or more carbons, and optionally terminating with or containing a cycloalkane or cyclic ether, (ii) a linear chain alkoxy alkane of the formula —(CH₂)_{q}O(CH₂)ᵣCH₃ where q is 1 to 5 and r is 1 to 15, and optionally terminating with or containing a cycloalkane in the r segment, or (iii) a chain containing a repeating sequence of (CH₂CH₂O)ₜ terminating with —OR² wherein t equals a value of 1 to 50 and R² is hydrogen or any linear alkane, or cyclic alkane, or any mixture thereof.

In some embodiments, R¹ is the same in all esterified n units. In other embodiments, R¹ is different in a plurality of the esterified n units. In most embodiments the copolymer is monoesterified and has greater than 10% or greater than 15%, or even greater than 25% total esterification.

In formula (IIa), r can be 1 or more carbons, more preferably 4, or more carbons. In some embodiments, r is 10 to 25, more preferably 10 to 20.

In formula (IIb), r can be any integer from 0 to 12. In some embodiments, r is 5 to 25, more preferably 8 to 20, and s is 0 to 9.

In formula (IIc), q is typically 1 to 5 and r is 1 to 15. In other embodiments, q is 2 to 5 and r is 5 to 15, or even 8 to 15. Several examples for —OR₁ are hexoxy ethoxylate, heptoxy ethoxylate, octoxy ethoxylate, decoxy ethoxylate, undecoxy ethoxylate, or dodecoxy ethoxylate. In an embodiment where —OR₁ is octoxy ethoxylate and the copolymer is about or greater than 50% monoesterified, the functionalized copolymer is stable in aqueous solution at a pH greater than 7.0 and at a magnesium ion concentration less than 10 mM. In an embodiment where R¹ is decoxy ethoxylate and the copolymer is greater than 30% monoesterified, the functionalized copolymer is stable in aqueous solution at a pH greater than 6.5 and at a magnesium ion concentration less than 10 mM.

In formula (IId), q is typically 1 to 5 and r is 1 to 15 and s is typically 0 to 9. In some embodiments, q is 2 to 5 and r is 5 to 15, or even 8 to 15.

In one embodiment, R¹ comprises the linear alkane chain and/or the linear chain alkoxy alkane, which can be partially or fully halogenated or partially or fully deuterated. Likewise, if a cyclic carbon chain is present on either of the linear alkane chain and/or the linear chain alkoxy alkane, the carbons here can also be partially or fully halogenated or partially or fully deuterated.

Referring now to formula (IIe), R¹ comprises the chain containing a repeating sequence of (CH₂CH₂O)ₜ terminating with —OR², the copolymer is monoesterified with greater than 20% total esterification, and t=1 to 50. R² can be hydrogen, any linear alkane, or cyclic alkane. When R² is a linear alkane, the number of carbon atoms can be 1 to 16. When R² is a cyclic alkane, the number of carbon atoms in the ring is 3 to 12. Several examples are diethylene glycol, triethylene glycol, tetraethylene glycol, pentaethylene glycol, and hexaethylene glycol. In an embodiment where —OR¹ is diethylene glycol and the copolymer is greater than 50% monoesterified, the functionalized copolymer is stable in aqueous solution at a pH greater than 5.0 and at a magnesium ion concentration up to 50 mM. In an embodiment where R¹ is tetraethylene glycol and the copolymer is greater than 40% monoesterified, the functionalized copolymer is stable in aqueous solution at a pH greater than 5.0 and at a magnesium ion concentration up to 100 mM.

In all embodiments, the modified DIBMA is generally unlimited in the ratio of 1 to m and in the size of n, which determines the molecular weight thereof. The average molecular weight (M_{w}) will typically be less than 500,000 daltons (Da), more particularly less than 150,000 Da. In some embodiments, the average molecular weight is less than 20,000 Da but greater than 1,500 Da. M_{w}/Mₙ(Mₙ being the number average molecular weight) indicates the polydispersity, and will typically be less than 10, more particularly less than 4, or even less than 3. In some embodiments, the polydispersity will be less than 2 (for example less than 1.5).

The 1 to m monomer ratio of diisobutylene to maleic acid can be 0.5:1 to 8:1. Exemplary monomer ratios herein are greater than 1:1 and can be any of those listed above when discussing SMA. In one embodiment of the invention the DIBMA has an average molecular weight in the range 2,000 to 12,000 and a ratio of diisobutylene to maleic acid of 1:1.

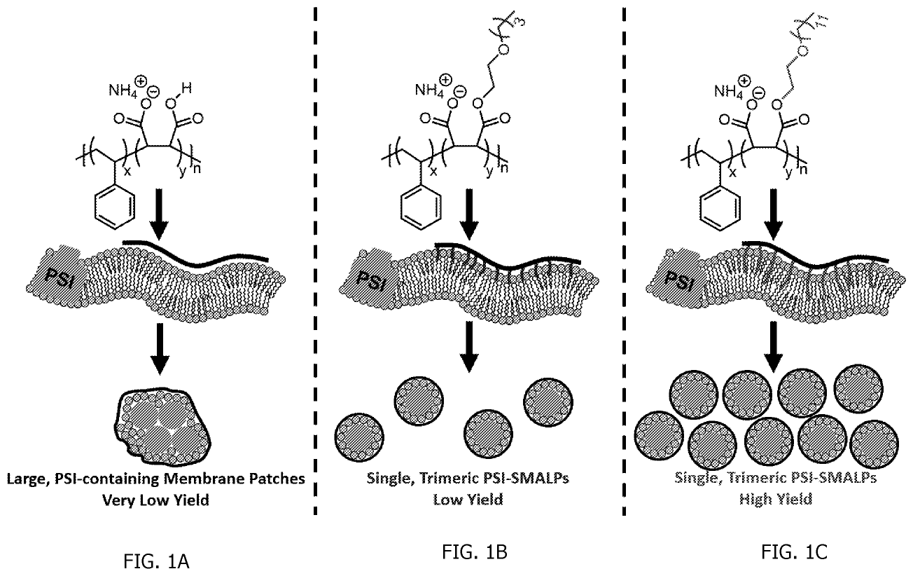

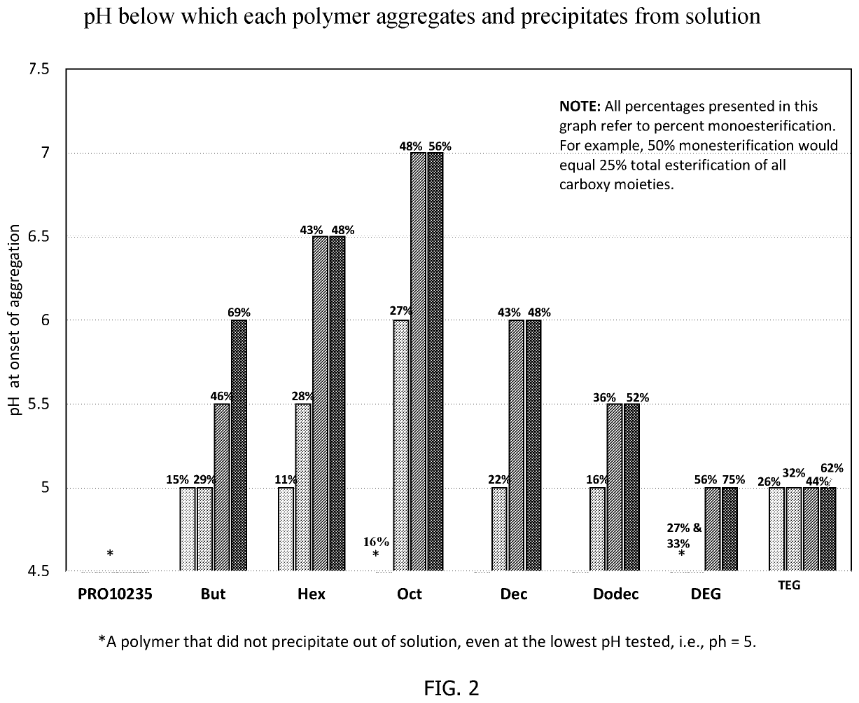

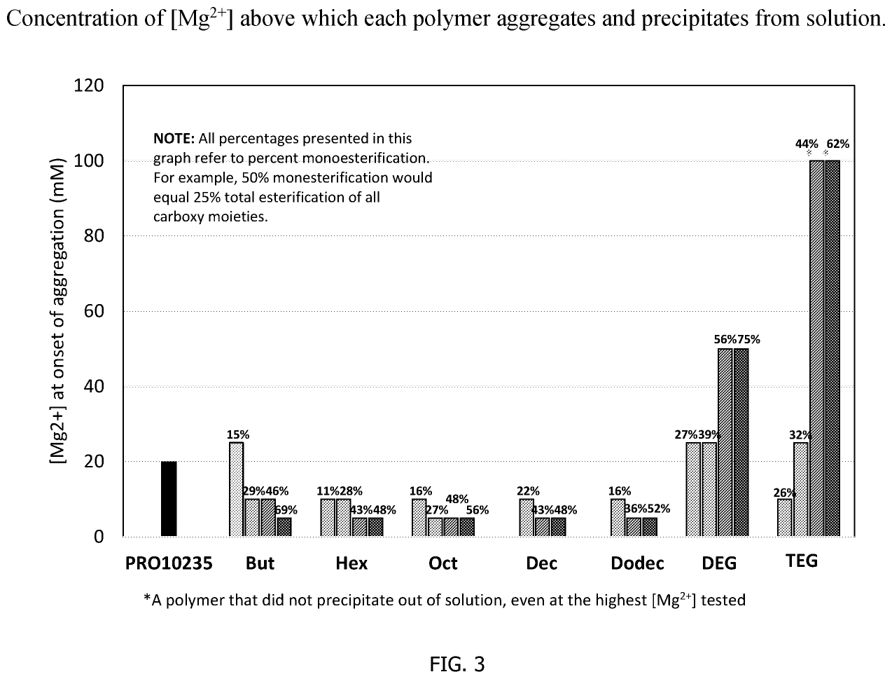

The modified SMA and modified DIBMA disclosed herein are useful for extracting lipids from membranes that are lipid-rich in the form a nanodisc shaped lipid particles. A representation of a SMA lipid particle is provided in FIG. 1 hereof and in FIG. 1 of Applicant's co-pending U.S. application Ser. No. 17/594,503, filed on Oct. 20, 2021.

Lipids suitable for extraction from biomolecules using the modified maleic acid copolymers disclosed herein will typically be membrane forming lipids. Membrane forming lipids comprise a diverse range of structures including galactolipids, phospholipids (some examples include the glycerophosholipids such as phosphatidylcholine, phosphatidylethanolamine, phosphatidylglycerol, phosphatidylinositol, phosphatidylserine, and cardiolipin; ether glycerol[ids such as plasmalogens and platelet activating factor), sphingolipis (some examples includes glycolipids such as cerebrosides, sulfatides, globosides, gangliosides, and other examples include sphingophospholipids such as sphingomyelin), and ceramides, and among others. Membrane forming lipids typically have a polar head group (which in a membrane aligns towards the aqueous phase) and one or more hydrophobic tail groups (which in a membrane associate to form a hydrophobic core). The hydrophobic tail groups will typically be in the form of acyl esters, which may vary both in their length (typically from 8 to 26 carbon atoms) and their degree of unsaturation (number of multiple bonds present). Phosphatidylcholine (PtdCho) and phosphatidylethanolamine (PtdEtn) heads are zwitterionic, whereas phosphatidylserine (PtdSer) and phosphatidylinositol (PtdIns) heads are anionic.

Sphingolipids (SL) contain one hydrophobic acyl chains and a phosphate head group ester linked to a Sph backbone. Their hydrophobic backbone is an ester or amide derivative of Sph with fatty acids being ceramide (Cer) the simplest representative. Sphingomyelin (SM) contains a phosphorylcholine headgroup associated to the sphingoid base. SM is the more abundant SL in the plasma membrane (PM) of mammalian cells. Within the total PL fraction of the PM, SM accounts for 2%-15% upon the cell type. Other SLs are glycosphingolipids (GSLs). GSLs are based on glucosylceramide (GlcCer) or on galactosylceramide (GalCer) and contain mono-, di- or oligosaccharides. Sphingolipids are defined by the presence of a sphingoid-base backbone (i.e., 2-aminoalk[ane or ene]1,3-diol with 2S,3R stereochemistry). The main feature that allows the formation of an impermeable lipid bilayer is the amphipathic nature of these molecules, resulting in a highly hydrophobic core and hydrophilic surface, the landmark of biological and model membranes.

Lipids suitable for extraction from biomolecules using the modified maleic acid copolymers disclosed herein may be of natural or synthetic origin, and may be a single pure component (e.g., 90% pure, especially 95% pure and suitably 99% pure on a weight basis), a single class of lipid components (for example, a mixture of phosphatidylcholines, or alternatively, a mixture of lipids with a conserved acyl chain type) or may be a mixture of many different lipid types. A single pure lipid is generally of synthetic or semi-synthetic origin. Examples of pure lipids include phosphatidylcholines and phosphatidylglycerols.

Mixtures of lipids are more typically of natural origin, obtained by extraction and purification by means known to one of skill in the art. Exemplary lipid extracts include: Epikuron 200 available from Degussa Texturant Systems UK Ltd; Emulmetik 950, Emulmetik 930, Pro-Lipo H and Pro-Lipo Duo available from Lucas Meyer Cosmetics SA; Liposome 0041, S 75, S 100, S PC, SL 80 and SL 80-3 available from Lipoid GmbH; Phospholipon® 90H, Phospholipon® 80H, Phospholipon® 90 NG, Nat 8539 available from Phospholipid GmbH. Lipid extracts of plant origin may typically be expected to demonstrate higher levels of unsaturation as compared to those of animal origin. It should be noted that, due to variation in the source, the composition of lipid extracts may vary from batch to batch.

### Working Example

General Materials and Methods. All chemical reagents were obtained from commercial sources and used without further purification, unless otherwise noted. 2-octyloxyethanol, 2-decyloxyethanol, and 2-dodecyloxyethanol were synthesized using a modified literature procedure. Kharlamov et al., Synthesis of some acyclic quaternary ammonium compounds: Alkylation of secondary and tertiary amines in a two-phase system, Russian Chemical Bulletin 2014, 63 (11), 2445-2454. PRO 10235 was obtained from Cray Valley (now Polyscope). One important note is that the specific batch of PRO 10235 used in this study was experimentally found to have a 1.21:1 ratio of styrene:maleic anhydride using ¹H NMR spectroscopy. Tetrehydrofuran (THF) was dried using an Innovative Technology Pure Solv solvent purification system. Water was purified using a MilliQ reverse osmosis system with a resistance of >18 MΩ to mitigate impact of trace ions. ¹H NMR spectroscopy was performed using a Varian 500 MHz NMR spectrometer and chemical shifts are reported with respect to residual solvent peaks.

General Synthesis of Alkoxy Ethoxylates. In a typical procedure, a mixture of KOH (9.26 g, 165 mmol) in ethylene glycol (42 mL, 750 mmol) was added to 1,4-dioxane (90 mL), Bu₄NBr (2.9 g, 9 mmol), and a 1-bromoalkane (150 mmol). The reaction mixture was then heated to 105° C. for 5 h. The mixture was cooled to room temperature and extracted with CHCl₃ (×3). The organic layer was separated, washed with water (×3), concentrated under vacuum, and the remaining residue purified via vacuum distillation to provide each product as a clear oil. Yields for 2-octyloxyethanol, 2-decyloxyethanol, and 2-dodecyloxyethanol were 60%, 64%, and 73%, respectively. The characterization of each product agreed with prior literature reports.

Esterification of PRO 10235. Esterification was performed using a modified literature procedure. Francisco Martinez et al., Monoesterification of Styrene-Maleic Anhydride Copolymers with Aliphatic Alcohols, Bol. Soc. Chil. Quim. 2001, 46. PRO 10235 (2.5 g, 9.8 mmol) was dissolved in dry THE (45 mL), followed by the addition of a 4-dimethylaminopyridine (DMAP) (0.06 g, 0.05 mmol) solution in dry THE (5 mL). The target alcohol (80 mmol) was then added to the polymer solution. The reaction was stirred under constant reflux for prescribed time intervals. Therein, about 8 mL aliquots were taken at various time points in order to achieve varying extents of esterification. To each aliquot was added HCl solution (10 mL, 0.001M). The organic layer was decanted, concentrated under vacuum, and dried in a vacuum oven at 80° C. for a minimum of 24 h.

Determination of Esterification Percentage. Utilizing the experimentally determined incorporation ratio of 1.21:1 styrene:maleic anhydride in PRO 10235, the extent of esterification was determined via ¹H NMR spectroscopy by comparing the integration of the aryl region to the proton signal corresponding to the methyl protons of the attached sidechain. DOSY NMR spectroscopy was also used to confirm functionalization of the polymer backbone with the desired alcohol, rather than free alcohol remaining in solution.

Solubilization of Prepared SMA Derivatives. The solubilization of the SMA derivatives was performed by placing the target SMA (15% w/v) in water (80% w/v) and adding a solution of 30% NH₄OH in water (5% w/v). Each solution was heated at 80° C. for ≥30 min, until a non-turbid solution was obtained.

Determination of pH and Divalent Cations Sensitivity in Aqueous Media. The solubility of each polymer sample as a function of pH was determined using a modified literature procedure. Scheidelaar et al., Effect of Polymer Composition and pH on Membrane Solubilization by Styrene-Maleic Acid Copolymers, Biophysical journal 2016, 111 (9), 1974-1986. Therein, each polymer sample was diluted into a standard Britton-Robinson (BS) buffer containing 150 mM NaCl for a final concentration of 0.15% (w/v). The prepared buffers ranged from pH 4.5 to 10, in half unit increments. The samples were then mixed via orbital shaking for 10 min and the optical density was measured at 350 nm using a UV spectrometer. Optical density values above the baseline were interpreted as an indicator of polymer aggregation and precipitation from solution.

Divalent cation sensitivity was also determined using a modified literature procedure from Burridge et al. noted in the background section above. Each polymer sample was diluted into a 9.5 pH tris buffer containing various concentrations of MgCl₂, ranging from 1 mM to 100 mM. The final concentration of each polymer solution was 0.15% (w/v). These solutions were then mixed via orbital shaking for 10 min and the optical density was measured at 350 nm using a UV spectrometer. The optical density values above the baseline were interpreted as an indicator of polymer aggregation and precipitation from solution.

Critical Aggregation Concentration Determination. The critical aggregation concentration (CAC) for the tested polymer samples was determined following a previous literature procedure. Scheidelaar et al., Effect of Polymer Composition and pH on Membrane Solubilization by Styrene-Maleic Acid Copolymers, Biophysical journal 2016, 111 (9), 1974-1986. Therein, each polymer sample was diluted to 0.15% (w/v) in a standard BR-buffer at a pH of 9.5. These polymer solutions were placed into a 96-well plate and each sample diluted 5-fold (×12) across the wells. A Nile Red solution was added to each well at a concentration of 1 μM. Each plate was excited at 550 nm and the emission was measured between 550-700 nm in 1 nm increments. The wavelength of the highest emission intensity was plotted versus concentration for each polymer sample. The CAC was determined by fitting sigmoidal curves to the blue shifting fluorescence spectra as polymer concentration increased.

Preparation of Thylakoid Membranes. Thylakoid membranes were isolated following established protocols discussed in Brady et al. (noted in the background section). Briefly, Te cells were grown in BG-11 media, in an air lift, flat panel bioreactor at 45° C. (Photon Systems Instruments, Brno, Czech Republic). The cells were irradiated with about 50 μmol photons mol⁻¹·cm⁻² of light from red and white LEDs and aerated with compressed air. The cells were harvested at late log phase, pelleted at 6,000 g and re-suspended in Tris-Cl (50 mM, pH 9.5, at room temperature), with KCl (125 mM) (Buffer S) to yield 1 mg/mL chlorophyll (Chl a) solutions. The cells were then incubated at 40° C. in Buffer S with 0.0025% (w/v) lysozyme (Gold Bio, United States) for 1 h in the dark, at 250 rpm on an orbital shaker. The intact cells were then pelleted at about 10,000 g for 10 min, re-suspended in Buffer S, and Dounce-homogenized. The cells were then mechanically lysed (×10) using a benchtop LM10 microfluidizer at 23,000 psi. The intact cells and debris material were pelleted at about 10,000 g for 10 min and discarded. The thylakoid membranes contained in the supernatant were then pelleted at about 190,000 g. This pellet was resuspended using a brush and was Dounce-homogenized in Buffer S (×3) to remove membrane-associated proteins. The resultant thylakoid membranes were then diluted to 1 mg/mL chlorophyll and stored at −20° C. prior to solubilization.

Membrane Solubilization and Protein Isolation using SMALPs. Thylakoid membrane aliquots (500 μL) were incubated with an alkoxy-functionalized SMA copolymer in the dark at a final concentration of 1.5% (w/v) for 3 h at 40° C., while shaking (250 rpm, orbital shaker). The samples were centrifuged at about 190,000 g for 15 min and the supernatant was removed using a flame-drawn Pasteur pipette. These supernatants, which contain PSI-SMALPs, were then analyzed directly or were further purified using sucrose density gradients and ultracentrifugation.

Sucrose Density Gradient Ultracentrifugation. The PSI-SMALP containing supernatants were then purified by sucrose density gradient centrifugation (SDGC) using a linear gradient of 10-30% (w/v) on a fixed 50% (w/v) sucrose cushion. Next, 1 mL of SMA-solubilized supernatant was loaded on top of the gradient and centrifuged in a SW-32 swinging bucket rotor for 20 h at about 150,000 g. The lowest green band was harvested using a needle syringe. This band has been shown to be PSI-SMALP.

Protein Analyses. Absorbance spectroscopy was performed using a dual-beam benchtop spectrophotometer (Evolution 300, ThermoScientific). Following solubilization and high-speed sedimentation, the supernatants were diluted (×20) prior to absorbance measurements. Chlorophyll was extracted with 90% methanol at 65° C. for 2 min and absorbance was taken at 665 nm to determine chlorophyll concentration. See Iwamura et al., Improved Methods for Determining Contents of Chlorophyll, Protein, Ribonucleic Acid, and Deoxyribonucleic Acid in Planktonic Populations, Internationale Revue der gesamten Hydrobiologie und Hydrographie 1970, 55 (1), 131-147 for methods of chloropyll extraction.

Sodium dodecyl sulfate-polyacrylamide gel (SDS-PAGE) analysis was performed using a sample solubilization buffer containing about 350 mM dithiothreitol and 4% SDS. The samples were heated in a 65° C. water bath for 9 min prior to loading onto a Bio-Rad TGX stain-free Criterion pre-cast gel. The gel was then illuminated and fixed prior to imaging of the TGX fluorochrome using a Bio-Rad ChemiDoc MP gel imaging system.

Low temperature fluorescence was performed at 77 K on presumed trimeric PSI bands following sucrose density gradient ultracentrifugation using methods from Cherepanov et al., PSI-SMALP, a Detergent-free Cyanobacterial Photosystem I, Reveals Faster Femtosecond Photochemistry, Biophysical Journal 2020, 118 (2), 337-351. These analyses were performed using a PTI Quantamaster dual-channel fluorimeter (HORIBA). Spectra are averaged over three measurements following background subtraction and normalized to the maximum fluorescence.

Transmission Electron Microscopy. PSI-SMALPS were isolated using sucrose density gradient ultracentrifugation. The isolated SMALP samples were diluted (×10-50) into Buffer S. A copper coated grid was placed in a 20 μL drop of the sample solution for 1 min. Excess sample was removed via filter paper absorption and the grid was washed by dipping it in distilled water. Excess distilled water was removed via filter paper absorption. The grid was then immediately stained by placing it in a 20 μL drop of 1% uranyl acetate for 1 min. The grid was allowed to dry and imaged using a JEOL 1440-Flash TEM at 200 kV.

### Results and Discussion

Prior to this disclosure it has been unknown how the extent of functionalization and identity of sidechains impact protein solubilization and specificity. Herein, monoesterification of an SMA polymer with hydrophobic alkoxy ethoxylate side-chains that have a length greater than butoxyethanol leads to increased solubilization efficiency of trimeric Photosystem I (PSI) from galactolipid membranes, such as membranes of the cyanobacterium Thermosynechococcus elongatus. The specific SMA polymer used herein, PRO 10235, is unable to encapsulate single PSI trimers from this Thermosynechococcus elongatus; however, as it is functionalized with alkoxy ethoxylates of increasing alkoxy chain length, a clear increase in trimeric PSI solubilization efficiency is observed. See the pictural representation of this in FIG. 1. And unexpectedly, an exponential increase in solubilization efficiency is observed when >50% of the maleic acid repeat units are monoesterified with long alkoxy ethoxylates. This suggests that the PSI extraction mechanism is highly dependent on both the number and length of the attached side-chains.

Synthesis and Characterization of SMA Derivates. PRO 10235, a commercially available copolymer of styrene and maleic anhydride, was functionalized with various alkoxy ethoxylates using DMAP-catalyzed esterification according to Scheme 1 below.

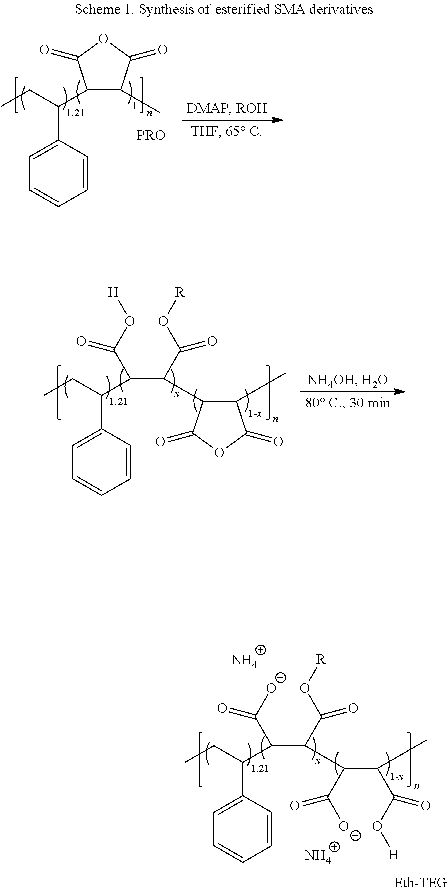

R of the R-functionalized SMA was tested with each of the following:

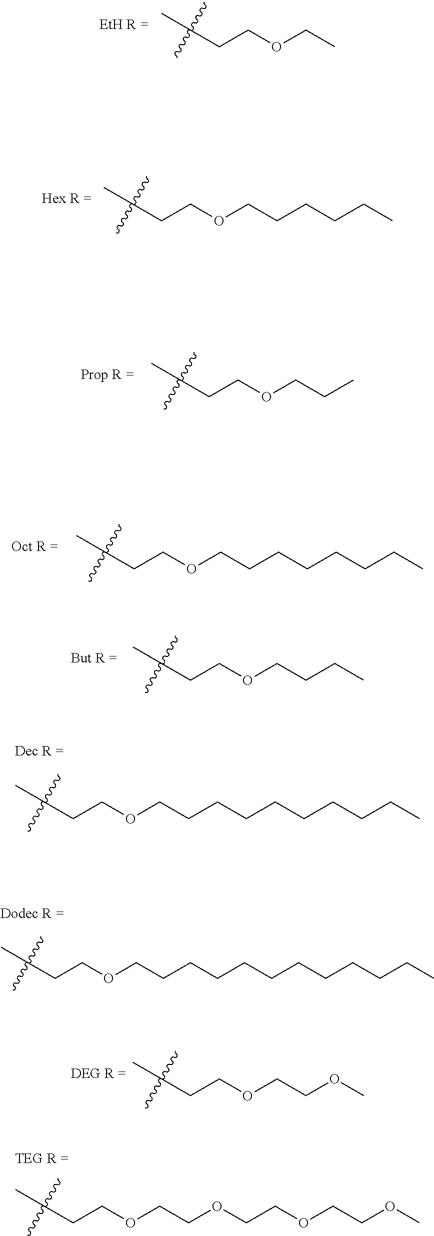

The diethylene glycol (DEG) or tetraethylene glycol (TEG) moieties were made to probe the role hydrophobicity of the alkoxy ethoxylates has on the lipid extraction process. For each esterification reaction, aliquots were taken as a function of reaction time to obtain polymer samples with increasing amounts of attached sidechains. These aliquots were purified via washing and dried in a vacuum oven to remove unreacted reagents and solvent. ¹H NMR spectroscopy was used to determine the extent of esterification for each sample. As a note, individual polymer identifiers will be used herein to identify a specific degree of esterification for each polymer sample. For example, polymer Eth with 16% monoesterified acid groups will be referred to as Eth-(16). 16% monoesterified is equivalent to 8% overall esterification.

Determination of pH Sensitivity. To determine how sidechain functionalization affects polymer solubility as a function of pH, each polymer was introduced to a series of BR-buffers ranging from pH=4.5 to 10, in half unit increments. As pH becomes more acidic, the polymers studied convert from their carboxylate forms to their carboxylic acid forms, thereby becoming insoluble in an aqueous medium and aggregation is observed. Thus, polymer solubility can be quantified by measuring changes in optical density at 350 nm. Referring to FIG. 2, the pH at which aggregation begins is presented as a bar graph. The unmodified PRO 10235 remained soluble at all pH values tested. For all SMA derivatives synthesized in this study, the solubility window decreased with increasing percent esterification. As expected, this effect is amplified when functionalized with more hydrophobic moieties (e.g., Eth to Dodec) as compared to more hydrophilic DEG/TEG substituted polymers, which remain soluble at lower pH values even as esterification percentage increased.

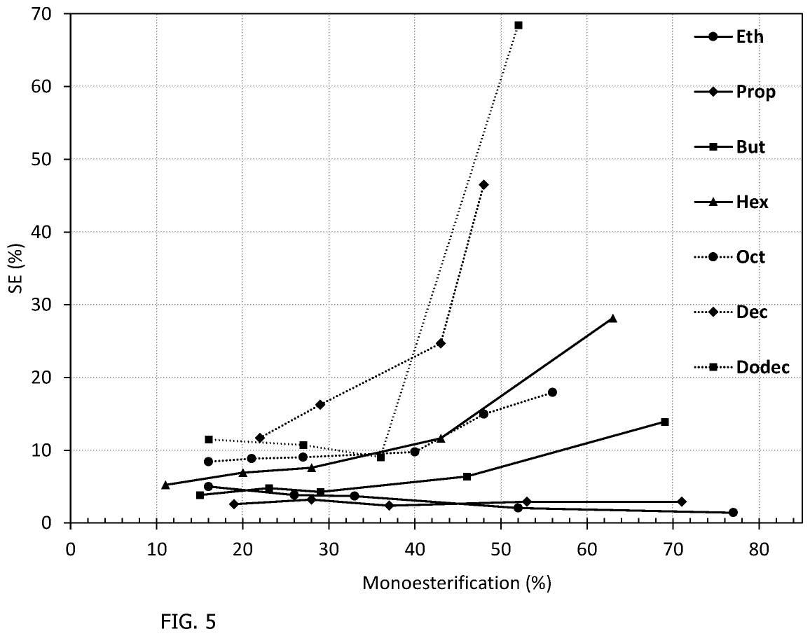

Determination of Divalent Cation Sensitivity. Another important characteristic is how well the synthesized SMA derivatives tolerate divalent cations. For this assay, each polymer was subjected to a series of pH=9.5 tris buffers containing increasing concentrations of MgCl₂, [Mg²⁺]. A graph of the values is presented as FIG. 3. Polymers Eth to Dodec were each found to be sensitive to divalent ions, with most beginning to aggregate in the presence of ≤10 mM MgCl₂. Furthermore, this divalent ion sensitivity increases with increasing percent esterification. In contrast, DEG and TEG polymer samples show a reverse trend, becoming more divalent ion tolerant at increasing degrees of esterification, and with TEG-(44) and TEG-(62) remaining soluble at 100 mM MgCl₂, the highest concentration tested. These sidechains contain ethereal units that are known to complex cations and promote solubility in aqueous environments.

Determination of Critical Aggregation Concentration. To determine the polymer concentration at which aggregation occurs, which is often referred to as critical aggregate concentration (CAC), we employed a previously described method using fluorescence spectroscopy and Nile Red as a reporter. Upon excitation at 550 nm, the fluorescence emission maximum of Nile Red blue shifts as it partitions into a hydrophobic environment. Therein, the extent of emission maximum blue shift corresponds to increased hydrophobicity in solution, which arises due to aggregation of the polymers in solution. As seen in FIG. 4, the plots of fluorescence wavelength maxima as a function of concentration yields a sigmoidal relationship that reveals aggregation for the highest percent esterified SMA copolymers occurs around 20-100 μg/mL, with the exception of TEG-(62) that aggregates around 0.6 to 3 mg/mL. Interestingly, we also observed that SMA derivatives functionalized with hydrophobic sidechains (Hex to Dodec) exhibited larger fluorescence maxima blueshifts than more hydrophilic polymers PRO and TEG-(62), agreeing with our hypothesis that aggregation of Hex to Dodec will generate a more hydrophobic environment for Nile Red partitioning.

Solubilization of Pigment-Protein Complexes from Te. To investigate protein extraction efficiency and selectivity, each alkoxy functionalized SMA copolymer derivative was incubated with isolated Te thylakoid membranes. This resulted in the solubilization of PSI. Following centrifugation, the solubilized PSI-complexes, which are pigmented with carotenoids and chlorophyll, remained in the supernatant while un-solubilized material sedimented. The supernatant was then analyzed using visible absorbance spectroscopy to qualitatively determine carotenoid and chlorophyll content. The peaks arising from carotenoids overlap in the spectral region between 425 nm and 550 nm, overlaying the Soret band for chlorophyll that is centered at 440 nm. Traces of phycocyanin (λ_{MAX}=about 625 nm), and a prominent chlorophyll absorbance peak at about 680 nm were also observed.

The alkoxy ethoxylate functionalized SMAs Prop to Dodec displayed an increasing qualitative trend of carotenoid release as a function of increasing percent esterification, as evidenced by an increase in absorbance. A similar qualitative trend was observed with regards to the amount of chlorophyll being liberated. While absorbance spectra are not fully quantitative, they enable us to make qualitative observations as to the effect that polymer functionalization has on their ability to liberate PSI, although while retaining the profiles of both carotenoids and chlorophyll within the complex. The increasing absorbances of both carotenoids and chlorophyll as a function of increasing polymer functionalization suggests that the amount of PSI extracted increases similarly and that these complexes retain their associated pigments following removal from the thylakoid membrane.

The DEG-functionalized SMA displayed the opposite qualitative trend in the solubilization of chlorophyll-containing proteins, wherein higher percentages of esterification appear to decrease the amount of chlorophyll-containing proteins being liberated from the Te membrane. The TEG-functionalized SMA showed no discernible solubilization trend at all. Absorbance profiles for the supernatants of SMA 1440 and DDM were measured as controls to ensure consistency across all trials. Lastly, the absorbance profile for the supernatant resulting from membrane extraction with DMAP showed very little absorbance between 400-800 nm, suggesting that potential for trace amounts of this reagent remaining in the synthesized polymer samples did not artificially impact our results.

While the DEG and TEG polymers were tested with the Te membrane, they are more suitable for solubilization of phospholipids from a phospholipid membrane rather than a galactolipid membrane. The Te membrane used in the above working examples is a galactolipid membrane.

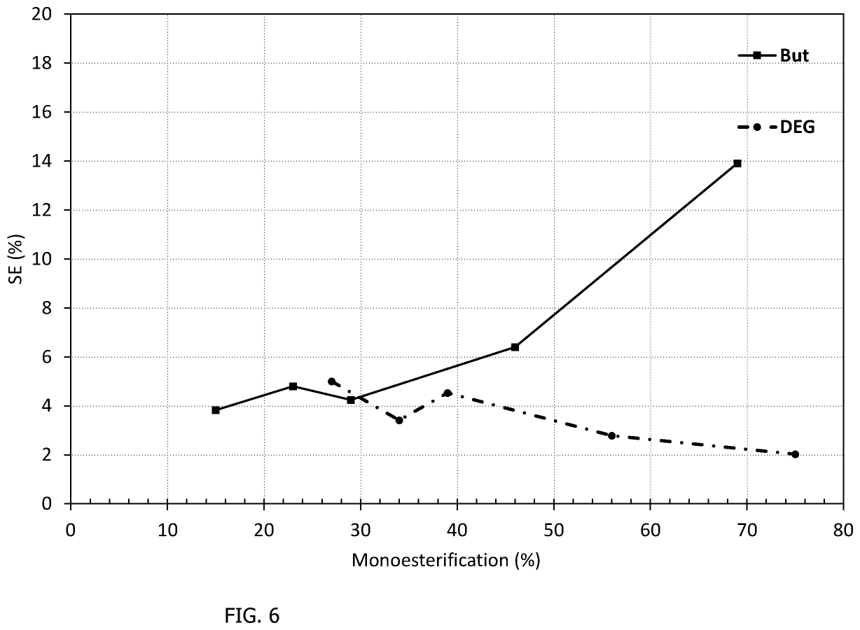

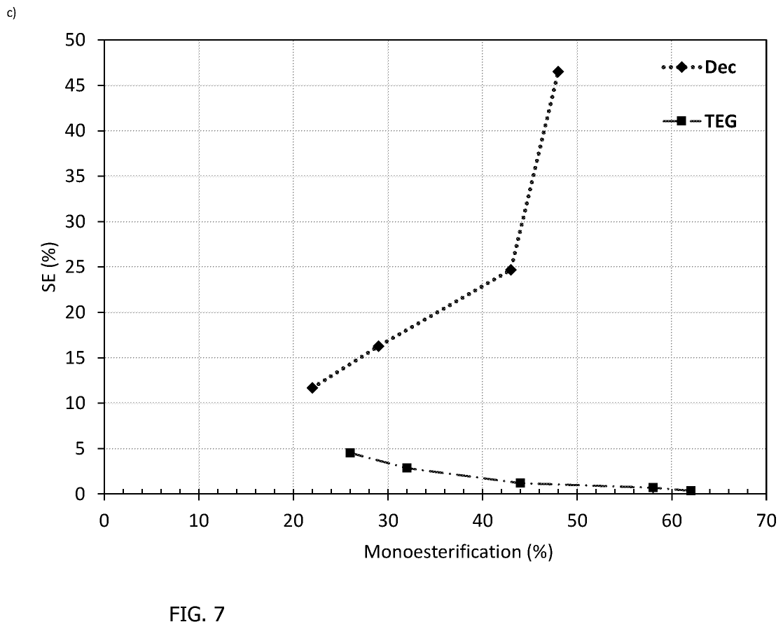

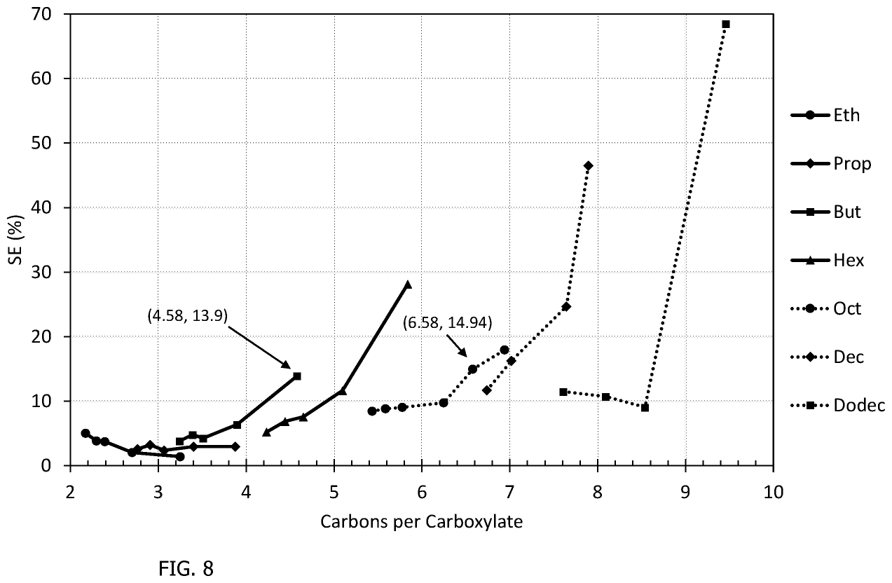

To quantify the percent solubilization efficiency (SE) of extracted chlorophyll proteins of each SMA formulation, we compared extracted chlorophyll in the supernatant to the 1 mg/mL chlorophyll of the starting thylakoid membrane and reported this as a percentage (% SE) as shown in the graph of FIG. 5. Quantification of this data confirms what was qualitatively observed: the longer alkoxy ethoxylate sidechains tend to elicit a higher solubilization efficiency than shorter alkoxy ethoxylates, especially as percent esterification increases. Furthermore, these trends suggest that sidechain length alone is not the primary factor resulting in increased solubilization efficiency. Rather, the increased hydrophobicity of the sidechain appears to have the most notable impact. This can be seen in FIGS. 6 and 7 by comparing alkoxy ethoxylate sidechains to ethylene glycol sidechains of similar lengths. For instance, polymers But and DEG both have six-atom sidechains. However, DEG is much more hydrophilic due to the additional ethereal units present. The same observation holds true for polymers Dec and TEG, which both contain sidechains thirteen atoms long, although TEG is more hydrophilic due to the attached tetraethylene glycol moiety. In both instances, the more hydrophilic polymer exhibits a markedly higher percent solubilization efficiency in the extraction of PSI from Te, particularly at high degrees of esterification.

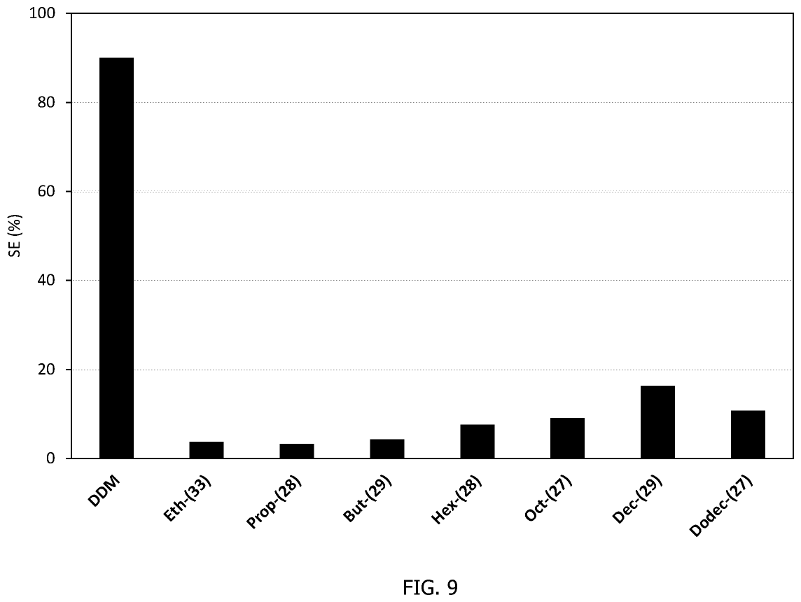

The trends observed in FIGS. 5-7 are informative, but it is difficult to draw direct comparisons relating hydrophobicity and % SE when the polymers studied bear varying extents of esterification as well as alkoxy moieties of differing length. As such, the data was normalized for polymers Eth to Dodec based upon their number of side-chain carbons per the average number of carboxylate moieties present in the polymeric repeat unit. Therein, the average number of carboxylates per repeat unit was calculated based upon a) percent esterification and b) knowing that unfunctionalized maleic acid units contain two carboxylates whereas monoesterified maleic acid units contain only one carboxylate. The number of carbons per alkoxylated ester (e.g., decyloxyethoxy units contain 14 carbons) was then divided by the average number of carboxylates per repeat unit to obtain a new metric that relates hydrophobic sidechain content to hydrophilic carboxylate content. Using such calculations, FIG. 8 is a normalization plot of solubilization efficiency as a function of the average number of carboxylates per maleic acid. This data indicates that both longer alkoxy ethoxylates and higher extents of esterification lead to higher solubilization efficiencies. As an example, though Oct-(56) and Dodec-(52) bearing octyloxy and dodecyloxy substituents, respectively, have very similar degrees of esterification, the latter features a solubilization efficiency (SE) that is about 400% higher than that of the former. Without being bound by theory, it is believed that the exponential increase in SE results from increased interactions between longer alkoxy ethoxylate sidechains and the tails of the galactolipids present in the thylakoid membrane of Te. Of particular interest are modified SMAs of formula I, in particular of formula (ii), that have a percent solubilization efficiency of trimeric Photosystem I (PSI) from membranes of the cyanobacterium Thermosynechococcus elongatus as a function of sidechain carbons per carboxylate that is greater than 14%.

Attaching longer alkoxy chains to SMA leads to a large decrease in aqueous solubility. This may be problematic when being used to solubilize membrane proteins that need water soluble polymers because Oct, Dec, Dodec can gelate in water when functionalized beyond about 55% monoesterification (overall 25% esterification). This suggests that though increasing chain length and degree of esterification appear to favor higher solubilization efficiencies, polymer samples functionalized with sidechains that change the balance of the hydrophobic and hydrophilic nature of portions of the copolymer structure may eventually become too unbalanced to remain in solution in an aqueous solution. On the contrary, SMAs of formula Ie, specifically those tested that were modified with DEG or TEG, were less hydrophobic modifications compared to those of formulas Ia-Id and may not gelate as readily in water soluble polymers, thereby making them more preferable when working with water soluble polymers.

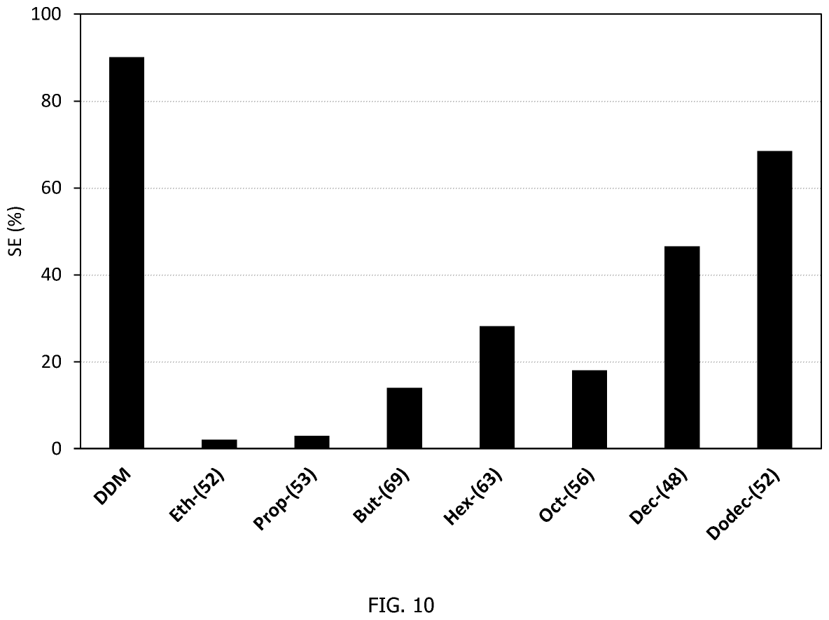

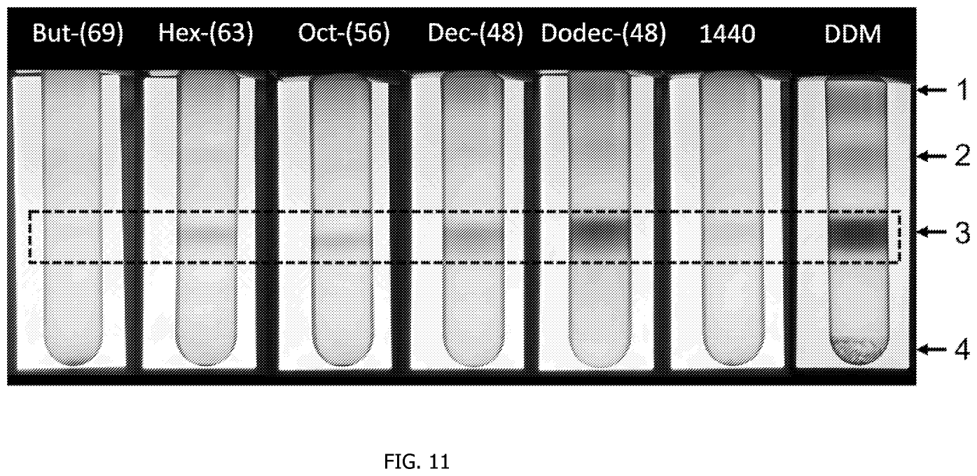

Another striking feature observed in FIG. 8, is that an empirical threshold of monoesterification was observed at about 50%, beyond which a drastic increase in percent SE is observed for polymers Hex, Dec, and Dodec. This effect is amplified as the length of the attached alkoxy ethoxylate increases, as illustrated by comparing polymer samples functionalized at both low and high degrees of monoesterification. As shown in FIG. 9, a bar graph shows that, when comparing samples featuring about 30% esterification of different alkoxy sidechains, the samples with longer sidechains exhibit slightly higher SE. In contrast, as shown in the bar graph of FIG. 10, SE increases significantly between samples as the degree of esterification is pushed past the empirical threshold of 50% esterification. For example, while the dodecyloxy substituted Dodec-(27) elicits an 80% increase in SE compared to the hexyloxy substituted Hex-(28), Dodec-(52) achieves a SE of 243% higher than Hex-(63).

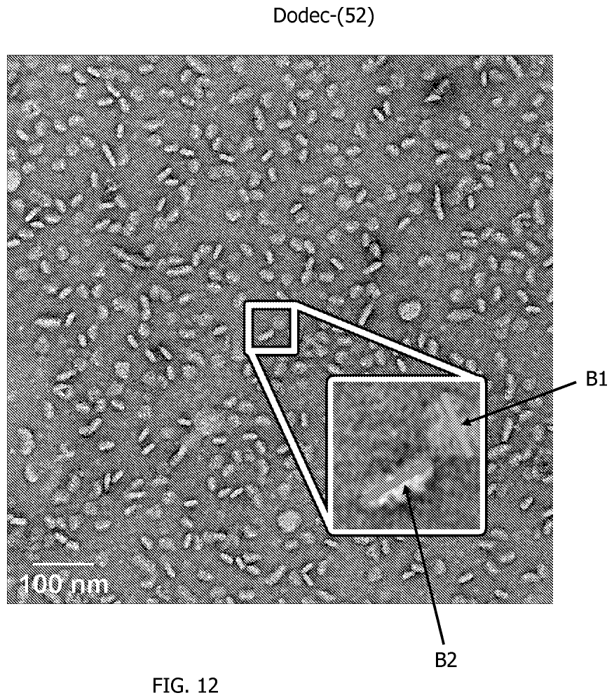

Characterization of Pigment-Protein Complexes Isolated from Te. To determine whether the chlorophyll extracted using the SMA derivatives reported herein is bound to protein complexes or free in solution, the supernatant following membrane solubilization was purified using a sucrose density gradient. FIG. 11 is a photograph of results for the sucrose density gradients of PSI-SMALPs following solubilization with alkoxy functionalized SMA copolymers. The black dashed box indicates the trimeric PSI band. Numbers to the right indicate the band number. As can be seen in FIG. 11, the top band is orange and contains liberated carotenoids (band 1). Band 2 is a diffuse green band that contains free chlorophyll, and, in the case of DDM, monomeric PSI and PSII. The trimeric PSI band, band 3, is noted in the black, dashed box and larger PSI particles (aggregates in the case of DDM) are seen at the bottom of the gradient, noted as band 4. Interestingly, samples with longer alkoxy ethoxylates resulted in high-density chlorophyll-containing fractions dissipating in favor of higher contents of SMALPs containing single PSI trimers. In the case of polymer Dodec, the larger chlorophyll-containing complexes are completely absent, see band 4 in FIG. 11. Some aggregated chlorophyll-containing protein complexes can be seen on the 50% sucrose (w/v) cushion at the bottom, band 4, of the DDM gradient in FIG. 11 (additional DDM was not included in this sucrose gradient buffer to maintain the dynamic equilibrium between DDM micelles and DDM bound the PSI toroid). For the purposes of this control, thereby rendering it acceptable, trimeric PSI is shown to be the dominant species arising from DDM isolation. Lastly, this side-by-side comparison allows us to observe that the SMALPs do not seem to require excess SMA in solution to maintain the integrity of the formed nanodiscs.

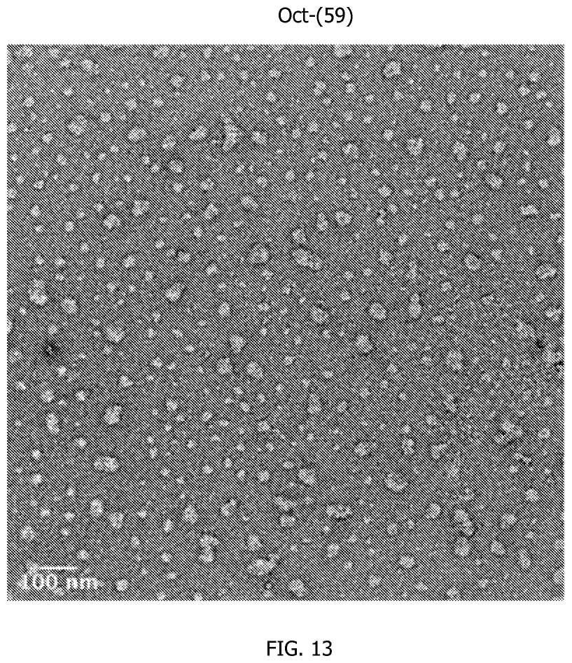

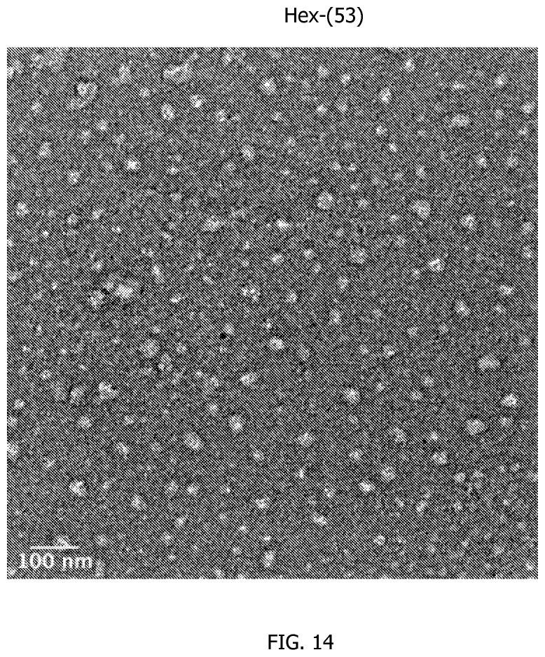

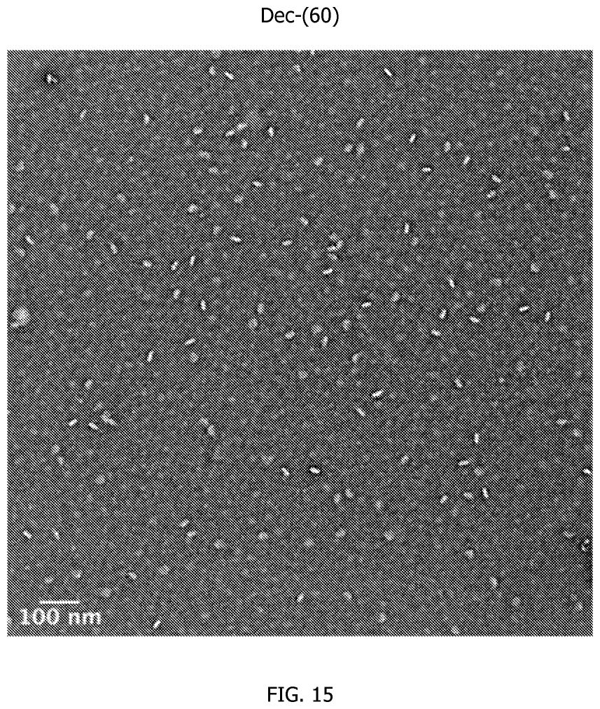

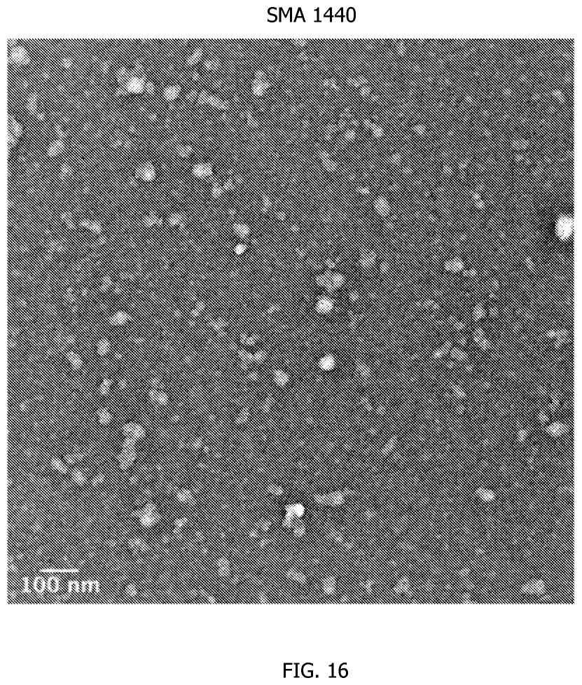

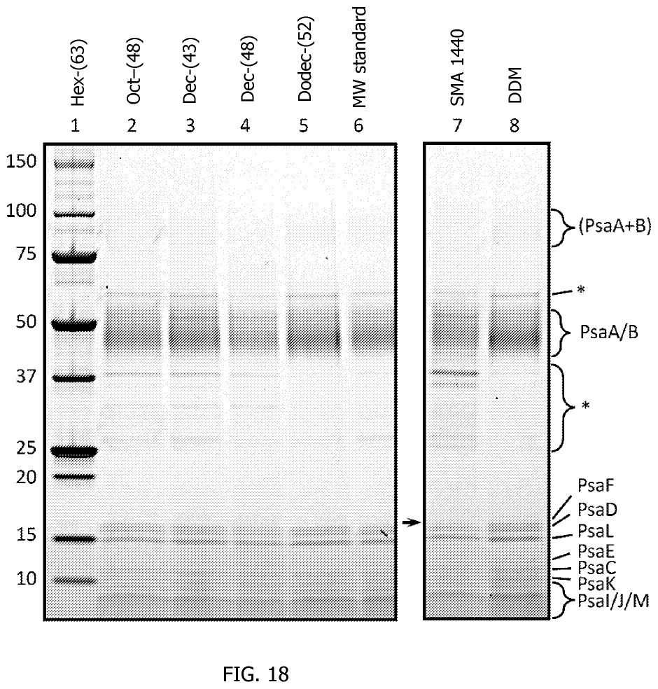

The trimeric PSI containing band 3 was harvested from each sucrose density gradient and was imaged using transmission electron microscopy (TEM) and a negative stain, with the exception of But-(69) due to low yield, to provide evidence of derivatized SMALP formation. FIG. 12 is a representative micrograph of Dodec-(52), which provides clear evidence of derivatized SMALP formation. The bars B1 and B2 depicted in the micrograph inset represent distances measured to determine average diameters. B1 depicts measurement across the face of a SMALP and B2 represents measurement along the side of a SMALP. The average diameter of the SMALPs (n=10) are listed in Table 1, wherein Oct-(59) was found to form the largest discs (FIG. 13), followed by Hex-(53) (FIG. 14), Dodec-(52) (FIG. 12), and Dec-(60) (FIG. 15). DDM is shown in FIG. 17. Interestingly this pattern correlates with the pH stability study and the maximum blue shifts recorded. These data may suggest that an optimum hydrophobicity may be achieved using SMA derivative Oct-(59), which allows this copolymer to stabilize larger lipid annuli around the trimeric PSI-SMALPs, while at the same time narrowing the solubility conditions of the polymer itself

TABLE 1
Average measured diameters of derivatized SMALPs.^{a}

| | Avg. | Standard | Standard |
|---|---|---|---|
| | Diameter | Deviation | Error |
| Solubilizing Agent | (nm) | (nm) | (nm) |
| DDM | 21.5 | 2.5 | 0.8 |
| 1440 | 34.7 | 5.4 | 1.7 |
| Hex-(53%) | 31.2 | 4.3 | 1.4 |
| Oct-(59%) | 35.4 | 6.1 | 1.9 |
| Dec-(60%) | 26.8 | 2.2 | 0.7 |
| Dodec-(52%) | 28.4 | 6.8 | 2.2 |

*Deviation and error are calculated based upon measurement of 10 SMALPs using ImageJ software.

The trimeric PSI fractions (band 3 in FIG. 11) were collected and separated using SDS-PAGE to determine their polypeptide profiles. Referring to the SDS-PAGE results provided in FIG. 18, the typical PSI profile was observed across all SMA copolymers tested. The * denotes unknown contaminants. PsaA+B represents the non-dissociated heterodimer. PsaA/B represents their respective monomers. The black arrow next to lane 7 points to the missing PsaF band in the SMA 1440. The peripherally associated PsaF subunit seems to be missing in the SMA 1440 control but not in the alkoxy functionalized SMAs synthesized herein or the DDM.

Low-temp fluorescence emission scans of the presumed PSI trimer containing fractions were performed to further confirm the formation of PSI containing SMALPs. Following excitation at 430 nm, free chlorophyll in solution exhibited a fluorescence at <680 nm, as well as a very minor peak at 695 nm signifying minimal extraction of PSII. The alkoxy ethoxylate functionalized PSI-SMALPs of this working example showed very little free chlorophyll/PSII and generally displayed an F_{MAX} of 728-729 nm. As such, modified maleic acid copolymer lipid particles were formed that comprise a lipid from a phospholipid rich membrane or a galactolipid rich membrane and any of the modified maleic acid copolymers disclosed herein. As seen in the above examples, in one embodiment, the lipid is from a galactolipid rich membrane of a cyanobacterium, but the membrane is not limited thereto. In the examples, the galactolipid-rich membrane was a cyanobacterium, more specifically, Thermosynechococcus elongatus.

Little is known about how altering the chemical composition of SMAs and SMA derivatives effects the efficiency and selectivity of membrane protein extraction. Herein, in one embodiment, the data shows that alkoxy ethoxylate esterified SMAs can be used to promote the solubilization of trimeric PSI from Te via formation of derivatized SMALPs. We observed that increasing the relative hydrophobicity of the amphiphilic copolymer leads to an overall increase in the amount of trimeric PSI extracted from cyanobacterium Te membranes. From these results, two main characteristics of functionalized SMA copolymers were identified to have large impacts on protein extraction. First, SMA derivatives bearing longer alkoxy ethoxylate sidechains, such as dodecyloxy substituted Dodec, tend to elicit higher solubilization efficiencies as compared to polymers bearing shorter alkoxy ethoxylate sidechains. Second, the number of attached sidechains appears to be an even more important factor. To highlight this feature, we observed a drastic increase in solubilization efficiency as the extent of polymer monoesterification surpassed 50% (an overall 25% esterification).

These two discoveries provide fundamental insight into the mechanism of SMA-facilitated protein solubilization in Te. We hypothesize that the aliphatic chains aid in SMALP formation by anchoring into the acyl chain region of the membrane, similar to the action of the styrene moiety of SMAs utilized in previous studies. Jamshad et al., Structural analysis of a nanoparticle containing a lipid bilayer used for detergent-free extraction of membrane proteins. Nano Research 2015, 8 (3), 774-789. Other groups have noted the possibility of amphiphilic polymers utilizing functionalized sidechains to intercalate within the acyl chains present in the tails of membrane lipids. Ball et al., Influence of DIBMA Polymer Length on Lipid Nanodisc Formation and Membrane Protein Extraction. Biomacromolecules 2021, 22 (2), 763-772. We suspect that our alkoxy ethoxylates are functioning in a similar manner, wherein longer alkyl chains may be able to more effectively anchor due to favorable interactions with the tails of the galactolipids composing the membrane. Also, we hypothesize, without being limited by our theory, that polymers featuring higher degrees of esterification may facilitate greater intercalation into the lipid membrane and form more stable SMALP particles. This could explain the drastic increase of solubilization observed when esterification percentages surpass the empirically observed 50% monoesterification threshold (an overall 25% esterification).

We have synthesized and characterized a new series of modified SMAs (MoSMAs) that have dramatically improved bioactivity. These MoSMAs have enhanced protein solubilization and can be used to generate a series of uniform lipid particles that can enable the non-detergent isolation of membrane proteins. This new material (MoSMAs) will have applications in the following non-limiting example industries: pharmaceutical, bioenergy, protein isolation, drug delivery, and food.

It should be noted that the embodiments are not limited in their application or use to the details of construction and arrangement of parts and steps illustrated in the drawings and description. Features of the illustrative embodiments, constructions, and variants may be implemented or incorporated in other embodiments, constructions, variants, and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

Having described the invention in detail and by reference to preferred embodiments thereof, it will be apparent that modifications and variations are possible without departing from the scope of the invention which is defined in the appended claims.

1. A modified maleic acid copolymer comprising:
a general formula I and having 1% to 90% total esterification of the maleic acid (MA); 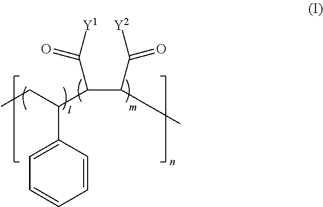 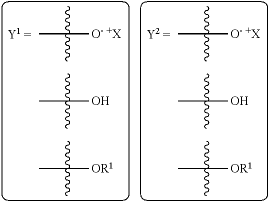
wherein R¹ is a moiety present in enough m units in one or both of Y¹ and Y² to provide a preselected percent of total esterification, and if not esterified are a hydrogen or a carboxylate unit with a counterion X⁺,
wherein R¹ comprises (i) a linear alkane chain having 11 or more carbons, (ii) a linear chain alkoxy alkane of the formula —(CH₂)_{q}O(CH₂)ᵣCH₃ where q is 1 to 5 and r is 1 to 15, with the proviso that when q=2, r is not 3, (iii) an alkane containing or terminating with a cyclic carbon chain, (iv) an alkoxy alkane containing or terminating with a cyclic carbon chain, (v) a chain containing a repeating sequence of (CH₂CH₂O)ₜ terminating with —OR² wherein t equals a value of 1 to 50 and R² is hydrogen, a linear alkane, or a cyclic alkane, or a mixture of (i) to (v);
1 and m have a ratio in a range of 1:1 to 8:1; and
n yields a copolymer having an average molecular weight of less than 500,000 daltons.

2. The modified maleic acid copolymer of claim 1, wherein R¹ comprises the linear alkane chain and/or the linear chain alkoxy alkane, which are partially or fully halogenated or is partially or fully deuterated.

3. The modified maleic acid copolymer of claim 1, wherein R¹ is the same in all esterified n units or is different in a plurality of the esterified n units.

4. The modified maleic acid copolymer of claim 1, wherein R¹ comprises the linear chain alkoxy alkane of the formula —(CH₂)_{q}O(CH₂)ᵣCH₃, q=1 to 5, and r=1 to 10, with the proviso that when q=2, r is not 3.

5. The modified maleic acid copolymer of claim 1, wherein the cyclic carbon chain of the alkane containing a cyclic carbon chain or of the alkoxy alkane containing a cyclic carbon chain is partially or fully halogenated or is partially or fully deuterated.

6. The modified maleic acid copolymer of claim 1, wherein R¹ comprises the chain containing a repeating sequence of (CH₂CH₂O)ₜ terminating with —OR².

7. The modified maleic acid copolymer of claim 6, wherein the copolymer is monoesterified with greater than 20% total esterification and t=1 to 10.

8. (canceled)

9. The modified maleic acid copolymer of claim 8, wherein R² is hydrogen.

10. The modified maleic acid copolymer of claim 1, wherein the copolymer is monoesterified with greater than 10% total esterification, and R¹ comprises (ii) and r is 5 to 15 or the copolymer is monoesterified with greater than 20% total esterification, and R¹ comprises (ii) and r is 9 to 15.

11. (canceled)

12. The modified maleic acid copolymer of claim 1, wherein X⁺ is selected from the group consisting of ammonium, lithium, sodium, and potassium ions.

13. The modified maleic acid copolymer of claim 1, wherein 1 and m have a ratio of 1.2:1.

14. A modified maleic acid copolymer comprising:
a general formula II and having 1 to 90% total esterification of the maleic acid (MA); 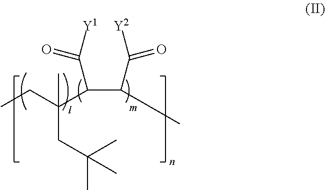 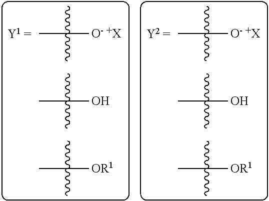
wherein R¹ is a moiety present in enough m units in one or both of Y¹ and Y² to provide a preselected percent of total esterification, and if not esterified are a hydrogen or a carboxylate unit with a counterion X⁺,
wherein R¹ comprises (i) a linear alkane chain, (ii) a linear chain alkoxy alkane of the formula —(CH₂)_{q}O(CH₂)ᵣCH₃ where q is 1 to 5 and r is 1 to 15, (iii) an alkane containing or terminating with a cyclic carbon chain, (iv) an alkoxy alkane containing or terminating with a cyclic carbon chain, (v) a chain containing a repeating sequence of (CH₂CH₂O)ₜ terminating with —OR² wherein t equals a value of 1 to 50 and R² is hydrogen, a linear alkane, or a cyclic alkane, or a mixture of (i) to (v);
1 and m have a ratio in a range of 0.5:1 to 8:1; and
n yields a copolymer having an average molecular weight of less than 500,000 daltons.

15. The modified maleic acid copolymer of claim 14, wherein R¹ comprises the linear alkane chain and/or the linear chain alkoxy alkane, which are partially or fully halogenated or is partially or fully deuterated.

16. The modified maleic acid copolymer of claim 14, wherein R¹ is the same in all esterified n units or is different in a plurality of the esterified n units.

17. The modified maleic acid copolymer of claim 14, wherein R¹ comprises the linear chain alkoxy alkane of the formula —(CH₂)_{q}O(CH₂)ᵣCH₃, q=1 to 5, and r=1 to 10.

18. The modified maleic acid copolymer of claim 14, wherein the cyclic carbon chain of the alkane containing a cyclic carbon chain or of the alkoxy alkane containing a cyclic carbon chain is partially or fully halogenated or is partially or fully deuterated.

19. The modified maleic acid copolymer of claim 14, wherein R¹ comprises the chain containing a repeating sequence of (CH₂CH₂O)ₜ terminating with —OR².

20. The modified maleic acid copolymer of claim 19, wherein the copolymer is monoesterified with greater than 20% total esterification and t=1 to 10.

21. (canceled)

22. The modified maleic acid copolymer of claim 19, wherein the copolymer is monoesterified with about 10 to 15% total esterification and t=2.

23. The modified maleic acid copolymer of claim 14, wherein the copolymer is monoesterified with greater than 10% total esterification, and R¹ comprises (ii) and r is 5 to 15 or the copolymer is monoesterified with greater than 20% total esterification, and R¹ comprises (ii) and r is 9 to 15.

24. (canceled)

25. A modified maleic acid copolymer lipid particle comprising a lipid from a phospholipid rich membrane or a galactolipid rich membrane and a modified maleic acid copolymer of claim 1.

26-27. (canceled)
